(19) United States
(12) Patent Application Publication
(10) Pub. No.: US 2014/0107380 A1
(43) Pub. Date: Apr. 17, 2014

(54) METHOD FOR PRODUCING BIODERIVED DIPROPYLENE AND TRIPROPYLENE GLYCOLS WITHOUT PROPYLENE OXIDE

(71) Applicant: ARCHER DANIELS MIDLAND COMPANY, Decatur, IL (US)

(72) Inventors: Paul D. Bloom, Forsyth, IL (US); Padmesh Venkitasubramanian, Decatur, IL (US)

(21) Appl. No.: 14/116,787

(22) PCT Filed: May 2, 2012
(86) PCT No.: PCT/US2012/036039

§ 371 Date: Nov. 11, 2013

Related U.S. Application Data
(60) Provisional application No. 61/484,830, filed on May 11, 2011.

(51) Int. Cl. C07C 41/01

(52) U.S. Cl. CPC C07C 41/01
USPC 568/679

(57) ABSTRACT

A method is provided for producing bioderived dipropylene and tripropylene glycols without using propylene oxide. The method utilizes a bioderived (mono)propylene glycol as a feed, and in one embodiment performs an acid-catalyzed condensation process to convert the bioderived propylene glycol to products including at least dipropylene glycol and preferably including tripropylene glycol as well. Wholly biobased dipropylene glycol and tripropylene glycol products and derivative products made therefrom are described, with compositions of matter including the wholly biobased dipropylene glycol and tripropylene glycol or a derivative thereof and describing uses of the various wholly biobased products or of the compositions including the wholly biobased products. Biobased polypropylene glycols may be made in the same manner.

The present invention is concerned with the production primarily of dipropylene and tripropylene glycols, such as are presently made in the hydration of propylene oxide to make monopropylene glycol (hereafter, simply “propylene glycol”).

Propylene glycol has conventionally been produced from petrochemical sources. Commercial production of petroleum-based or—derived propylene glycol involves the hydration of propylene oxide, made predominantly by the oxidation of propylene. Propylene in turn is a product of the fossil fuels industry, for example, from fluid cracking of gas oils or steam cracking of hydrocarbons.

The world's supply of petroleum is, however, being depleted at an increasing rate. As the available supply of petroleum decreases or as the costs of acquiring and processing the petroleum increase, the manufacture of various chemical products derived therefrom (such as propylene glycol and ethylene glycol) will be made more difficult. Accordingly, in recent years much research has taken place to develop a suitable biobased propylene glycol product, which can be interchangeable with propylene glycol deriving from petroleum refining and processing methods but which is made from renewable versus nonrenewable materials.

As a result of these efforts, processes have been developed by several parties involving the hydrogenolysis of especially five and six carbon sugars and/or sugar alcohols, whereby the higher carbohydrates are broken into fragments of lower molecular weight to form compounds which belong to the glycol or polyol family. Sugars containing five carbon chains, such as ribose, arabinose, xylose and lyxose, and corresponding five carbon chain sugar alcohols such as xylitol and arabinitol, are among the materials contemplated in U.S. Pat. No. 7,038,094 to Werpy et al., for example, as are six carbon sugars such as glucose, galactose, maltose, lactose, sucrose, allose, altrose, mannose, gulose, idose and talose and six carbon chain sugar alcohols such as sorbitol. Some of these carbohydrate-based feedstocks are commercially available as pure or purified materials. These materials may also be obtained as side-products or even waste products from other processes, such as corn processing. The sugar alcohols may also be intermediate products produced in the initial stage of hydrogenating a sugar.

For other known examples of such processes, U.S. Pat. No. 5,206,927 describes a homogeneous process for hydrocracking carbohydrates in the presence of a soluble transition metal catalyst to produce lower polyhydric alcohols. A carbohydrate is contacted with hydrogen in the presence of a soluble transition metal catalyst and a strong base at a temperature of from about 25° C. to about 200° C. and a pressure of from about 15 to about 3000 psi. However, as is evident from Tables II and Ill in the disclosure of U.S. Pat. No. 5,206,927, about 2-7% of other polyol compounds are produced in the hydrocracking process. U.S. Pat. No. 4,476,331 describes a two stage method of hydrocracking carbohydrates using a modified ruthenium catalyst. European Patent Applications EP-A-0523 014 and EP-A-0 415 202 describe a process for preparing lower polyhydric alcohols by catalytic hydrocracking of aqueous sucrose solutions at elevated temperature and pressure using a catalyst whose active material comprises the metals cobalt, copper and manganese. Still other examples of such carbohydrate-based processes may be found without difficulty by those skilled in the art.

U.S. Pat. No. 6,455,742 to Cortright et al. describes a different, renewable source-based approach to making 1,2-propanediol/propylene glycol, wherein lactic acid—such as may be produced by fermentation of glucose—is catalytically hydrogenated to propylene glycol in the presence of a copper on silica catalyst.

Still other efforts have been based on the use of another readily accessible biobased feedstock, namely, glycerol. Glycerol is currently produced as a byproduct in making biodiesel from vegetable and plant oils, through the transesterification reaction of lower alkanols with higher fatty acid triglycerides to yield lower alkyl esters of higher fatty acids and a substantial glycerol byproduct. Glycerol is also available as a by-product of the hydrolysis reaction of water with higher fatty acid triglycerides to yield soap and glycerol. The higher fatty acid triglycerides may derive from animal or vegetable (plant) sources, or from a combination of animal and vegetable sources as well known, and a variety of processes have been described or are known.

In the context of vegetable oil-based biodiesel production and soap making, all sorts of vegetable oils have been combined with the lower aliphatic alcohols or water. Preferred vegetable oils include, but are not limited to, soybean oil, linseed oil, sunflower oil, castor oil, corn oil, canola oil, rapeseed oil, palm kernel oil, cottonseed oil, peanut oil, coconut oil, palm oil, tung oil, safflower oil and derivatives, conjugated derivatives, genetically-modified derivatives and mixtures thereof. As used herein, a reference to a vegetable oil includes all its derivatives as outlined above. For instance, the use of the term “linseed oil” includes all derivatives including conjugated linseed oil.

A biobased glycerol is also available as a product of the hydrogenolysis of sorbitol, as described in an exemplary process in U.S. Pat. No. 4,366,332, issued Dec. 28, 1982.

U.S. Pat. Nos. 5,276,181 and 5,214,219 thus describe a process of hydrogenolysis of glycerol using copper and zinc catalyst in addition to sulfided ruthenium catalyst at a pressure over 2100 psi and temperature between 240-270° C. U.S. Pat. No. 5,616,817 describes a process of preparing 1,2-propanediol (more commonly, propylene glycol) by catalytic hydrogenolysis of glycerol at elevated temperature and pressure using a catalyst comprising the metals cobalt, copper, manganese and molybdenum. German Patent DE 541362 describes the hydrogenolysis of glycerol with a nickel catalyst. Persoa & Tundo (Ind. Eng. Chem. Res. 2005, 8535-8537) describe a process for converting glycerol to 1,2-propanediol by heating under low hydrogen pressure in presence of Raney nickel and a liquid phosphonium salt. Selectivities toward 1,2-propanediol as high as 93% were reported, but required using a pure glycerol and long reaction times (20 hrs). Crabtree et al. (Hydrocarbon processing February 2006 pp 87-92) describe a phosphine/precious metal salt catalyst that permit a homogenous catalyst system for converting glycerol into 1,2-propanediol. However, low selectivity (20-30%) was reported. Other reports indicate use of Raney copper (Montassier et al. Bull. Soc. Chim. Fr. 2 1989 148; Stud. Surf. Sci. Catal. 41 1988 165), copper on carbon (Montassier et al. J. Appl. Catal. A 121 1995 231)), copper-platinum and copper ruthenium (Montassier et al. J. Mol.. Catal. 70 1991 65). Still other homogenous catalyst systems such as tungsten and Group VIII metal-containing catalyst compositions have been also tried (U.S. Pat. No. 4,642,394). Miyazawa et al. (J. Catal. 240 2006 213-221) & Kusunoki et al (Catal. Comm. 6 2005 645-649) describe a Ru/C and ion exchange resin for conversion of glycerol in aqueous solution. Still other examples of like processes may be found without difficulty by those skilled in the art.

One downside to all of the aforementioned biobased PG processes, however, lies in the fact that dipropylene and tripropylene glycols (DPG and TPG, respectively) are not co-produced with the 1,2-propanediol/monopropylene glycol, as they are in the conventional petroleum-based chemistry based on the hydration of propylene oxide (“PO”).

By way of further background, in commercial processes for the hydration of PO, the reaction is typically uncatalyzed and carried out at around 200 degrees Celsius and under 15 atmospheres of pressure. DPG, TPG and minor quantities of higher alcohols are produced alongside the targeted PG product, and the proportion of PG to higher glycols is controlled by the molar ratio of PO to water in the initial reaction mixture; usually about 15 moles of water are used per mole of PO to optimize PG production. Presently, about 10 to 13 metric tons of DPG and from 1 to 3 metric tons of TPG are made per 100 metric tons of PG by this method. After evaporation of excess water, the PG, DPG and TPG are separated by distillation.

While the demand for PG is much larger as compared to the demand for DPG or TPG, yet DPG—which can also be produced according to a known reaction of PO and PG—and TPG have some market value themselves for various end uses. DPG, for example, is used for specialty benzoate ester plasticizers and plasticizer blends as a phthalate alternative, as well as in caulks, sealants, adhesives and resilient flooring. DPG also finds use as a low-odor solvent to both extract and carry fragrances and flavors, as well as in juice and soft drink applications, as an agricultural solvent, in brake fluids and other functional fluid formulations, in combination with other glycols in unsaturated polyester resins, in the preparation of alkyd resins using DPG as a substitute for a more expensive polyhydric polyol such as pentaerythritol, as a starting material for higher molecular weight polyols consumed in polyurethanes and in dipropylene glycol acrylates as an alternative to hexanediol systems. TPG for its part has been used for tripropylene glycol acrylates, for polyurethanes, for solvent/lubricant/textile soap applications and for plasticizers.

For offering a true biobased, “drop in” replacement to the petroleum-based slate of PG and PG-related and -derivative products, one would ideally be able to produce and offer biobased DPG and TPG products (and DPG- and TPG-related and derivative products, such as benzoate ester plasticizers based on DPG or TPG or acrylates based on DPG or TPG for example) in addition to a biobased PG. And while DPG could be made by reacting a biobased PG such as made by the above-described processes with propylene oxide, these DPG and TPG products would more preferably be made without using propylene oxide, so that DPG, TPG and related products may be made using or based on entirely renewable resources and may be wholly biobased.

Parenthetically, as these terms are used interchangeably herein, we intend by “biologically derived”, “bioderived” or “biobased” that these will be understood as referring to materials whose carbon content is shown by ASTM D 6866, in whole or in significant part (for example, at least 20 percent or more), to be derived from or based upon biological products or renewable agricultural materials (including but not limited to plant, animal and marine materials) or forestry materials. In this respect ASTM Method D6866, similar to radiocarbon dating, compares how much of a decaying carbon isotope remains in a sample to how much would be in the same sample if it were made of entirely recently grown materials. The percentage is called the biobased content of the product. Samples are combusted in a quartz sample tube and the gaseous combustion products are transferred to a borosilicate break seal tube. In one method, liquid scintillation is used to count the relative amounts of carbon isotopes in the carbon dioxide in the gaseous combustion products. In a second method, 13C/12C and 14C/12C isotope ratios are counted (14C) and measured (13C/12C) using accelerator mass spectrometry. Zero percent 14C indicates the entire lack of 14C atoms in a material, thus indicating a fossil (for example, petroleum based) carbon source. One hundred percent 14C, after correction for the post-1950 bomb injection of 14C into the atmosphere, indicates a modern carbon source. ASTM D 6866 effectively distinguishes between biobased materials and petroleum derived materials in part because isotopic fractionation due to physiological processes, such as, for example, carbon dioxide transport within plants during photosynthesis, leads to specific isotopic ratios in natural or biobased compounds. By contrast, the 13C/12C carbon isotopic ratio of petroleum and petroleum derived products is different from the isotopic ratios in natural or bioderived compounds due to different chemical processes and isotopic fractionation during the generation of petroleum. In addition, radioactive decay of the unstable 14C carbon radioisotope leads to different isotope ratios in biobased products compared to petroleum products.

For reducing dependence on the fossil fuels industry and on materials made or deriving from nonrenewable, fossil fuel sources, again, it would be preferable if dipropylene glycol and tripopylene glycol products could be made which are completely biobased and derive completely from renewable sources.

The present invention meets these and other needs by providing, according to a first aspect, a method for producing bioderived dipropylene and tripropylene glycols (together with other useful products) without using propylene oxide. The method utilizes a bioderived (mono)propylene glycol (CAS #57-55-6) as a feed, and in one embodiment performs an acid-catalyzed condensation process to convert the bioderived propylene glycol to products including at least dipropylene glycol (CAS #25265-71-8) and preferably including tripropylene glycol (CAS #24800-44-0) as well. The present invention in other respects concerns wholly biobased dipropylene glycol and tripropylene glycol products and derivative products made therefrom, compositions of matter including the wholly biobased dipropylene glycol and tripropylene glycol or a derivative thereof and uses of the various wholly biobased products or of the compositions including the wholly biobased products. In a further refinement, biobased polypropylene glycols (CAS #25322-69-4) can also be made starting from the bioderived propylene glycol, again without requiring the use of propylene oxide. In still a further refinement, a portion of the propylene glycol may be converted to propanal (propionaldehyde, CAS 123-38-6), which may then be used according to commonly-assigned U.S. patent application Ser. No. 61/484,834, “Processes for Making Acrylic-Type Monomers and Products Made Therefrom”, filed concurrently herewith, to produce acrylic acid and/or acrylate ester monomers and particularly biobased acrylic acid and/or acrylate ester monomers and related compositions.

As described above, dipropylene glycol (DPG) and tripropylene glycol (TPG) conventionally have been made as co-products in the hydration of propylene oxide to make monopropylene glycol (PG, or 1,2-propanediol), with a certain fairly well-defined and limited range of amounts of both being produced according to the amount of water used in the hydration. The present invention is directed in a first, primary aspect to providing means for making both biobased DPG and TPG, and thus enabling the same mix of products to be made by a producer of biobased PG as would be made by a producer of a conventional nonrenewable, fossil fuels industry-dependent PG—but also enabling greater or lesser amounts of DPG and/or TPG to be made than would be realized in a conventional process based on the hydration of propylene oxide, should demand for DPG and/or TPG relative to PG change. In this regard, while some current applications for DPG and TPG have already been mentioned above, those skilled in the art will readily appreciate that demand for DPG and/or TPG could materially increase in a number of applications wherein a biobased alternative would be favored were one available at a comparable cost and with comparable performance attributes.

Further, while the present invention is thus mainly focused on enabling the manufacture of biobased DPG and TPG as an alternative to DPG and TPG from propylene oxide, the process of the present invention also does enable the production of useful biobased polypropylene glycols (PPGs) as well as other useful materials such as propanal.

By way of background, such PPGs are polymers of propylene glycol in various generally lower to medium range molecular weights, and these are also presently made from propylene oxide, through the base-catalyzed, anionic ring-opening polymerization of propylene oxide using an initiator with one hydroxyl group (which could be a monoalcohol or simply water), two hydroxyl groups (e.g., ethylene glycol) or three or more hydroxyl groups (glycerol, sorbitol, pentaerythritol, as examples).

PPGs have similar attributes and are used in many of the same applications as the polyethylene glycols. Certain PPGs are used as rheology modifiers in formulations for polyurethanes, as dispersants, surfactants and wetting agents in leather finishing, or as preferred base stocks for spin finish lubricants for fiber and textile processing generally. PPGs have diverse other uses, being a primary ingredient in the manufacture of paintballs, in toothpastes to prevent bacterial breakdown of the pyrophosphates used to control tartar buildup, and for sterilizing or pasteurizing nutmeats. PPGs are also widely used as defoamers, for example in textile processing applications, in fermentation foam control, in direct, indirect and secondary food additives, in water and wastewater treatment and papermaking operations. Finally, PPGs are used in metalworking applications, including in buffing and polishing compounds, cutting and grinding fluids, and lubricants for metal stamping, rolling and forming, as well as in heat transfer fluids, as wetting agents and dispersants in agricultural formulations, and as chemical intermediates—for example, reacting with acrylic acid or methacrylic acid to produce reactive monomers for radiation curable coatings or being epoxidized to produce resins used in coating applications where flexibility is needed. Still other uses and applications may be considered by those skilled in the art.

Fundamentally, the present invention enables the propylene oxide-independent production of biobased DPG, TPG and PPGs through an acid-catalyzed condensation of bioderived propylene glycol at elevated temperatures. The process can be carried out using a variety of acid catalysts, both solid heterogeneous acid catalysts that can be separated out and recovered for reuse by filtration or the like, as well as liquid acid catalysts. In the former category are catalysts on supports such as hydroxyapatite, phosphated silica and phosphotungstic acid supports. Strong mineral acid catalysts may be used, as well as weaker inorganic and organic acids. Examples are given below of several acid catalysts, and of acid catalysts in both solid and liquid forms. Preferred catalysts are phosphoric acid, trifluoroacetic acid, and tungstated zirconia.

The process can be performed on a batchwise, semi-batch or continuous basis. Where DPG, TPG and/or PPGs are principally of interest, and propanal (and the products derivable from propanal by following the teachings of the commonly-assigned, concurrently filed application) is generally of lesser interest, then it is expected that a continuous liquid phase, trickle bed reaction will be preferred. Where propanal formation is also important, then a batchwise or continuous fixed bed, vapor phase process is to be preferred. In a batchwise process, the propanal formed through dehydration of propylene glycol will condense with propylene glycol present to form 4-methyl-2-ethyl-1,3 dioxolane. The dioxalane can be isolated through distillation, for example, and hydrolyzed back to propylene glycol and propanal in water. The propanal may then be separated from a recycle propylene glycol solution by distillation, and further processed according to the commonly-assigned, concurrently filed application as discussed below. In a fixed bed, vapor phase process, propylene glycol can be converted to propanal in high yields without forming the dioxalane (and requiring a subsequent hydrolysis step to recover the propanal) by keeping the localized concentration of propanal to propylene glycol low—whether by dilution of the propylene glycol feed, by limiting the catalyst contact time and/or limiting the gas hourly space velocity (GHSV) in the reactor.

Catalyst loadings, reaction temperatures and reaction or residence times may vary somewhat based on a desire to favor greater production of DPG over TPG and PPGs, or TPG in preference to DPG and PPGs or of PPG, however, those skilled in the art will be well able to select conditions which are favorable to producing more or less of DPG, TPG and PPGs with the guidance provided herein, without undue further experimentation. In general, however, for favoring the production of DPG and TPG over PPGs, catalyst loadings on the order of five percent or less by weight based on propylene glycol are contemplated as preferred, and more preferably will be two percent or less, most preferably a half percent or less. Reaction temperatures from 135 degrees Celsius to 200 degrees Celsius are preferred, though more preferably will be from 175 to 189 degrees Celsius. Reaction or average residence times should preferably be less than 0.75 hours, more preferably less than 0.5 hours and most preferably 0.38 hours or less. Operating pressure for a trickle bed reactor should preferably be less than 1500 psi, gauge, more preferably less than 1000 psi, gauge, most preferably will be 500 psi, gauge or less.

The process of the present invention may also produce propanal, which as mentioned previously may be used to produce biobased acrylic acid and methacrylate ester monomers. As related in the commonly-assigned U.S. Patent Application Ser. No. 61/484,834 which has been filed concurrently herewith for “Processes for Making Acrylic-Type Monomers and Products Made Therefrom”, processes for chemically, catalytically dehydrating propylene glycol to propanal had been proposed, see, e.g., Applied Catalysis A: General, vol. 366, pp. 304-306 (2009)(silica-supported silicotungstic acid catalyst), Cheng, L. and Ye, X., Catalysis Letters, vol. 130, nos. 1-2, pp 100-107 (2009) (silicotungsten catalysts) and Lehr, V. et al., Catalysis Today, vol. 121, nos. 1-2, pp. 121-129 (2007)(bivalent transition metal sulfates in supercritical water), as had various enzymatic processes, see, e.g., using dioldehydrase (Frey, P. et al., J. Biological Chemistry, vol. 241, no. 11, pp. 2732-3 (1966); Zagalak, B. et al., J. Biological Chemistry, vol. 241, no. 13, pp. 3028-35 (1966); Abeles R. et al., J. American Chemical Society, vol. 93, no.5, pp. 1242-51 (1971)); glycerol dehydratase (Zheng, Y. et al., “Preparation of Lactobacillus Reuteri Glycerol Dehydratase and Its Use for Preparation of Aldehydes”, Faming Zhuanli Shenging Gongkai Shuomingshu (2010): Chinese Published Patent Application CN 2009-10153309, published Oct. 15, 2009); and propanediol dehydratase (US Patent Application No. 2010185017A, published Jul. 22, 2010).

In the previously referenced commonly-assigned, co-filed application, biobased propylene glycol undergoes an acid-catalyzed dehydration in the presence of the same catalysts as used to produce DPG, TPG and/or PPGs, and produces propanal. This propanal may then be used according to any of several process embodiments described in the commonly-assigned application, to make renewable source-based acrylic acid monomers and methacrylate ester monomers—which can be used as the corresponding, conventional nonrenewable source-based monomers, in acrylic acid, acrylic acid ester and methacrylate ester polymers and copolymers.

Briefly summarized, according to a first embodiment, the propanal so made is desaturated to form propenal, and the propenal is the oxidized to form the acrylic acid. Methyl methacrylate in a second embodiment may be made by subjecting the propanal to base-catalyzed aldol condensation to form methacrolein, and the methacrolein subjected to oxidative esterification to yield methyl methacrylate. In a third embodiment, methyl methacrylate may be made by subjecting the propanal to oxidative esterification to form methyl propionate, and the methyl propionate undergoes the base-catalyzed aldol condensation to methyl methacrylate. Details for carrying out these various embodiments can be found in the incorporated commonly-assigned application, and accordingly need not be further elaborated in the present application.

The present invention is illustrated more particularly by the non-limiting examples which follow:

### EXAMPLE 1

A 500 mL round bottom flask equipped with a magnetic stir bar, Dean-Stark trap and condenser was charged with 20 grams of propylene glycol and 1 gram of sulfuric acid (in the form of concentrated sulfuric acid). The reaction mixture was heated using an oil bath to 150 degrees Celsius. An organic-water azeotrope collected in the Dean-Stark trap as time passed, and when the trap was full it was emptied. The reaction mixture was heated for 5 hours, and became a dark brown oily liquid. After 5 hours, heating was stopped and the dark brown oily residue was sampled for analysis by gas chromatography. That analysis showed that 48.80 percent of the residue by weight was still propylene glycol, while 7.6 percent by weight was dipropylene glycol and 0.69 percent was tripropylene glycol. No effort was made for this experiment to specifically identify other materials included in the remainder.

### EXAMPLE 2

For this example, the same apparatus and steps were followed as in Example 1, except that the reaction mixture was heated to 180 degrees Celsius. Again, 20 grams of propylene glycol and 1 gram of sulfuric acid were used. The results of this higher temperature run as indicated by gas chromatographic analysis were that the residue included 61.4 percent by weight of propylene glycol, 8.1 percent by weight of dipropylene glycol and 0.69 percent of tripropylene glycol. No effort was again made to identify other materials which may have been included in the residue.

### EXAMPLE 3

For this example, the initial reaction mixture included 300 grams of propylene glycol and 15 grams of sulfuric acid, and the bath temperature was set to 180 degrees Celsius. The same procedure was followed as in Examples 1 and 2. Analysis of the residue by GC showed 14.2 percent of propylene glycol, 10.62 percent of dipropylene glycol and 5.68 percent of tripropylene glycol. No attempt was made to specifically identify other materials in the residue.

### EXAMPLE 4

A larger, 1 L Autoclave engineer reactor was charged with 400 grams of propylene glycol and 2 grams of sulfuric acid. The reactor system was assembled, pressurized to 500 psi with argon and heated to 200 degrees Celsius. The reactor was maintained at this temperature for 5 hrs, and then cooled to room temperature. The reaction mixture was transferred to a 1 L Pyrex bottle and submitted for analysis by gas chromatography/mass spectroscopy. The GC-MS results showed the product mixture as having 44.76 weight percent of propylene glycol, 9.75 weight percent of dipropylene glycol and 1.18 weight percent of tripropylene glycol. Propanal and dioxolane were also determined to be produced. While an accurate quantification of propanal and dioxolane cannot be made on the basis of the GC-MS chromatogram, relative area percentages of the various products identified by GC-MS can be reported and are provided in Table 1 following the last example.

### EXAMPLE 5

The 1 L Autoclave reactor was charged for this Example with 400 grams of propylene glycol and 4 grams of sulfuric acid, then pressurized with argon, heated to temperature, cooled and the product mixture analyzed as in Example 4. The analysis showed 36.5 weight percent of propylene glycol in the product mixture, with 9.8 weight percent of dipropylene glycol, and 2.02 weight percent of tripropylene glycol.

### EXAMPLE 6

The same apparatus and procedure were used as in Example 1, except that concentrated phosphoric acid was used to supply 15 grams of phosphoric acid to act on 300 grams of propylene glycol. The reaction mixture was heated to 190 degrees Celsius with the oil bath, beginning to reflux at about 160 degrees Celsius and accumulating an organic/water azeotrope in the Dean-Stark trap as before. After 5 hrs, heating was stopped, and the residue collected after cooling for analysis. The analysis showed 85.5 percent propylene glycol in the residue, with 6.58 weight percent of dipropylene glycol and 1.18 weight percent of tripropylene glycol.

### EXAMPLE 7

For this example, 4 grams of phosphoric acid were combined in the 1 L reactor with 400 grams of propylene glycol. Using the same procedure and the same conditions as in Examples 4 and 5, the resultant product mixture was found to contain 82.01 weight percent of propylene glycol, 3.64 weight percent of dipropylene glycol and 0.89 weight percent of tripropylene glycol.

### EXAMPLE 8

Concentrated phosphoric acid was again used, to supply 25 grams of phosphoric acid to the 1 L reactor with 500 grams of propylene glycol. The reactor in this case was again heated to 200 degrees Celsius and maintained at this temperature for 5 hours, but no pressurizing argon gas was used. After the reactor contents cooled to room temperature and were transferred for analysis, it was determined that the product mixture included 64.86 weight percent of propylene glycol, 9.83 weight percent of dipropylene glycol and 4.54 weight percent of tripropylene glycol.

### EXAMPLE 9

Example 8 was reproduced, except that the reaction temperature was changed from 200 degrees Celsius for five hours to 220 degrees Celsius for five hours. The product mixture included 41.01 weight percent of propylene glycol, 12.35 weight percent of dipropylene glycol and 3.57 weight percent of tripropylene glycol.

### EXAMPLE 10

Following the procedure of Examples 4 and 5, again, 400 grams of propylene glycol were input to the 1 L reactor with 8.4 grams of trifluoroacetic acid. The reactor system was assembled, pressurized to 500 psi with argon and heated to 200 degrees Celsius. The reactor was maintained at this temperature for 5 hrs, and then cooled to room temperature. The product mixture was transferred to a 1 L Pyrex bottle, and submitted for analysis. The product mixture was determined to contain 73.11 weight percent of propylene glycol, 5.88 weight percent of dipropylene glycol, and 0.29 weight percent of tripropylene glycol.

### EXAMPLE 11

The 1 L reactor was charged with 400 grams of propylene glycol and 15.3 grams of trifluoroacetic acid, assembled without addition of the argon gas, and heated to 200 degrees Celsius. After 5 hrs at this temperature, the reactor was allowed to cool to room temperature. Sampling and analysis of the reactor's contents showed propylene glycol at 68.45 percent by weight, 6.57 percent of dipropylene glycol, and 0.38 percent of tripropylene glycol.

### EXAMPLE 12

The 1 L reactor was charged with 400 grams of propylene glycol and 2 grams of methanesulfonic acid, assembled, pressurized with 500 psi of argon, heated to 200 degrees Celsius and maintained at this temperature for 5 hrs. At the conclusion of the 5 hrs, the reactor was allowed to cool to room temperature, then the products were sampled for analysis. Propylene glycol was 53.24 percent by weight of the product mixture, dipropylene glycol was 8.76 percent of the mixture, and tripropylene glycol was 0.81 percent of the mixture.

### EXAMPLE 13

The same apparatus, procedure and conditions were used as in Example 12, except that 4 grams of methanesulfonic acid were supplied to the reactor. The resultant product mixture contained 50.6 percent by weight of propylene glycol, 9.03 percent by weight of dipropylene glycol, and 0.97 percent by weight of tripropylene glycol.

### EXAMPLE 14

Twenty grams of a tungstated zirconia (XZ01250, batch no. PRB738, MEL Chemicals, Flemington, N.J.) catalyst was activated in a tube furnace at 650 degrees Celsius, ramped at 5 degrees Celsius_per minute to 700 degrees Celsius, over a period of 4 hours. The thus-activated catalyst was cooled to 250 degrees Celsius and added to 400 grams of propylene glycol. The 1 liter Autoclave engineer reactor was charged with this mixture, the reactor was assembled and heated to 180 degrees Celsius. The reactor was maintained at this temperature for 5 hours, cooled to room temperature and the product mix transferred to a 1 liter Pyrex bottle for analysis as in previous examples. The resultant product mixture contained 91.1 percent by weight of propylene glycol, 0.19 percent by weight of dipropylene glycol, and less than 0.01 percent by weight of tripropylene glycol. Any amounts of propanal or dioxolane which may have been produced were below detection limits.

TABLE 1
Relative area percentages for various
components detected by GC-MS

| | | | Propylene | Dipropylene |
|---|---|---|---|---|
| | Propanal | Dioxalane | glycol | glycol |
| Ex. 4 | 2,439,573 | 568,741,713 | 1,110,998,493 | 456,195,717 |
| Ex. 5 | 76,213,207 | 1,943,781,414 | 3,671,305,525 | 2,980,564,343 |
| Ex. 7 | 22,067,399 | 1,560,472,042 | 5,830,240,315 | 1,203,841,390 |
| Ex. 8 | 76,746,406 | 3,098,945,311 | 6,053,154,155 | 3,584,747,899 |
| Ex. 9 | 76,746,406 | 907,716,380 | 1,304,117,359 | 559,277,157 |
| Ex. 10 | 8,107,201 | 1,978,014,623 | 5,808,174,405 | 2,453,504,890 |
| Ex. 11 | 8,020,915 | 2,034,778,021 | 5,644,990,375 | 2,689,548,596 |
| Ex. 12 | 1,400,638 | 411,556,820 | 1,274,384,656 | 415,289,560 |
| Ex. 13 | 1,338,226 | 289,710,095 | 1,153,600,288 | 401,378,662 |
| Ex. 14 | ND | 238,979,805 | 5,724,798,487 | 80,467,526 |

### EXAMPLE 15

A 500 mL flame dried round bottom flask was charged with 100 grams of propylene glycol (freshly distilled), 100 mL of trifluoroacetic acid, 1.13 grams of p-toluene sulfonic acid and 40 grams of activated molecular sieves (3A). The flask was equipped with a reflux condenser and heated to 90 degrees Celsius. After 5 hrs of continuous reflux, the reaction mixture was diluted with dichloromethane. The molecular sieves were removed by filtration and the filtrate was vacuum concentrated, then the concentrate was subjected to short path distillation. A colorless liquid distilled overhead when the temperature was about 70 degrees Celsius and the pressure was kept to 3-5 mm Hg. This colorless liquid product was identified as propylene glycol trifluoroacetate by GC-MS analysis, whereas the residue was a viscous liquid identified as polypropylene glycol.

What is claimed is:

1. A process for producing bioderived dipropylene and tripropylene glycols, comprising causing a bioderived monopropylene glycol to undergo a condensation reaction at elevated temperatures and in the presence of an acid catalyst, whereby products including at least bioderived dipropylene and tripropylene glycols are produced.

2. A process according to claim 1, wherein the products also include polypropylene glycols.

3. A process according to either of claim 1 or 2, wherein the products also include 4-methyl-2-ethyl-1,3-dioxolane.

4. A process according to claim 3, further comprising separating out at least some of the 4-methyl-2-ethyl-1,3-dioxolane by distillation, hydrolyzing at least a portion of the 4-methyl-2-ethyl-1,3-dioxolane to propanal and monopropylene glycol in water, and recycling monopropylene glycol to the start of the process.

5. A process according to any of claims 1-3, wherein the products also include propanal.

6. A process according to any of claims 1-5, wherein the acid catalyst is selected from hydroxyapatite, phosphated silica and phosphotungstic acid catalysts, is a strong mineral acid, an inorganic or organic acid.

7. A process according to any of claims 1-6, performed on a batchwise, semi-batch or continuous basis.

8. A process according to claim 7, performed as a continuous, liquid phase trickle bed process.

9. A process according to any of claims 1-8, wherein the acid catalyst is used at five percent or less by weight based on the monopropylene glycol starting material.

10. A process according to claim 9, wherein the acid catalyst is used at two weight percent or less based on the monopropylene glycol.

11. A process according to claim 10, wherein the acid catalyst is used at 0.5 weight percent or less based on the monopropylene glycol.

12. A process according to any of claims 1-11, conducted at a temperature of from 135 degrees Celsius to 200 degrees Celsius, a pressure of less than 1500 psig and with an average residence time of less than 0.75 hrs.

13. A process according to claim 12, conducted at a temperature of from 175 degrees Celsius to 189 degrees Celsius, a pressure of less than 1000 psig and with an average residence time of less than 0.5 hrs.

14. A process according to claim 13, conducted at a temperature of from 175 degrees Celsius to 189 degrees Celsius, a pressure of less than 500 psig and with an average residence time of less than 0.38 hrs.

15. Bioderived dipropylene glycol.

16. Bioderived tripropylene glycol.

17. Bioderived polypropylene glycol.

18. A mixed bioderived glycols composition including bioderived dipropylene, tripropylene and polypropylene glycols.
